(19) United States
(12) Patent Application Publication
(10) Pub. No.: US 2022/0189580 A1
(43) Pub. Date: Jun. 16, 2022

(54) TRAIT PREDICTION MODEL GENERATION APPARATUS, TRAIT PREDICTION APPARATUS, AND METHOD FOR GENERATING A TRAIT PREDICTION MODEL

(71) Applicant: KABUSHIKI KAISHA TOSHIBA, Tokyo (JP)

(72) Inventors: Masahiro OZAWA, Yokohama Kanagawa (JP); Chenyuan XU, Kawasaki Kanagawa (JP); Kosuke HARUKI, Tachikawa Tokyo (JP)

(73) Assignee: KABUSHIKI KAISHA TOSHIBA, Tokyo (JP)

(21) Appl. No.: 17/521,023

(22) Filed: Nov. 8, 2021

(30) Foreign Application Priority Data
Dec. 10, 2020 (JP) 2020-205213

(51) Int. Cl. G16B 20/40; G06F 30/20; G16B 5/20; G16B 20/20

(52) U.S. Cl. CPC G16B 20/40; G16B 20/20; G16B 5/20; G06F 30/20

(57) ABSTRACT

According to one embodiment, a trait prediction model generation apparatus generates a plurality of first trait prediction models for each of a plurality of populations, based on summary statistics and inter-polymorphism correlated information. The apparatus generates a second trait prediction model for a specific one of the populations based on regularized regression of the first trait prediction models of each of the populations using a plurality of data sets including single-nucleotide polymorphism data and a trait value.

### CROSS-REFERENCE TO RELATED APPLICATION

This application is based upon and claims the benefit of priority from prior Japanese Patent Application No. 2020-205213, filed Dec. 10, 2020, the entire contents of which are incorporated herein by reference.

### FIELD

Embodiments described herein relate generally to a trait prediction model generation apparatus, a trait prediction apparatus, and a method for generating a trait prediction model.

### BACKGROUND

Genome-wide association studies (GWAS) are conducted as genetic statistical studies to comprehensively examine the association between tens of millions of genetic mutations existing on human genome sequences and the onset of human diseases. Polygenic risk scores obtained by calculating the weighted sum of genetic mutations for each individual using results of the genome-wide association studies, correlate with various diseases and traits. The GWAS is expected to be applied to personalized medicine according to the individual's constitution, such as preventive care to individuals at high risk of disease.

A number of methods have been studied to generate a prediction model that predicts the susceptibility of an individual to a disease, etc., from each individual's genomic data using comprehensive (genome-wide) summary statistics of the association between single nucleotide polymorphisms and traits obtained from genome-wide association analysis (see non-patent documents 1 (Nature: Common polygenic variation contributes to risk of schizophrenia that overlaps with bipolar disorder) and non-patent documents 2 (The American Journal of Human Genetics: Modeling Linkage Disequilibrium Increases Accuracy of Polygenic Risk Scores)).

In these methods, only the statistically significant single nucleotide substitutions that are useful for predicting traits are retained from the genome-wide summary statistics, and the value of the statistic is used as the prediction weight, for the single nucleotide substitution, or the value of the statistic is modified and used as the prediction weight. In addition, it is known that the prediction accuracy of the prediction models generated by these methods tends to improve as the sample size (number of subjects) of genome-wide association analysis increases.

However, these methods assume that the ethnic group for which the genome-wide association analysis is conducted and the ethnic group for which the prediction is to be made are the same, and it has been pointed out that, the prediction accuracy decreases for different ethnic groups (non-patent document 3 (Nature Genetics: Clinical use of current polygenic risk scores may exacerbate health disparities)).

Although genome-wide association analyses have been conducted in many parts of the world, most of these analyses have been conducted on Europeans, and there are no large-scale genome-wide association analysis results for non-Europeans such as Japanese. Therefore, if non-Europeans such as the Japanese are the target of prediction, prediction models generated based on the results of genome-wide association analysis of the same ethnic group will have only small prediction accuracy due to the small sample size. A prediction model based on the results of genome-wide association analysis of Europeans will have only a small prediction accuracy due to the influence of ethnic differences.

### BRIEF DESCRIPTION OF THE DRAWINGS

FIG. 1 is a block diagram showing an example of a configuration of a trait prediction model generation apparatus according to a first embodiment.

FIG. 2 is a flowchart showing an example of a process of the trait prediction model generation apparatus according to the first embodiment.

FIG. 3 is a diagram showing an example of single-nucleotide polymorphism data.

FIG. 4 is a schematic view showing an example of a configuration of a second trait prediction model.

FIG. 5 is a bar chart showing the prediction accuracy of four different trait prediction models according to an example of the first embodiment.

FIG. 6 is a block diagram showing an example of a configuration of a trait prediction apparatus according to the first embodiment.

FIG. 7 is a flowchart showing an example of a process of the trait prediction apparatus according to the first embodiment.

FIG. 8 is a schematic diagram of the example of a process of the trait prediction apparatus shown in FIG. 7.

FIG. 9 is a block diagram showing an example of a configuration of a trait prediction model generation apparatus according to a second embodiment.

FIG. 10 is a flowchart showing an example of a process of the trait prediction model generation apparatus according to the second embodiment.

FIG. 11 is a schematic diagram showing a process of dividing a genomic region in FIG. 10.

FIG. 12 is a block diagram showing an example of a configuration of a trait prediction apparatus according to the second embodiment.

FIG. 13 is a flowchart showing an example of a process of the trait prediction apparatus according to the second embodiment.

FIG. 14 is a schematic diagram of the example of a process of the trait prediction apparatus shown in FIG. 13.

### DETAILED DESCRIPTION

According to one embodiment, a trait prediction model generation apparatus includes a processing circuit. The processing circuit generates a plurality of first trait prediction models for each of a plurality of populations, based on summary statistics and inter-polymorphism correlated information. The processing circuit generates a second trait prediction model for a specific one of the populations based on regularized regression of the first trait prediction models of each of the populations using a plurality of data sets including single-nucleotide polymorphism data and a trait value.

The present inventors have found out that there is a difference in the method for generating an optimum prediction model between generation of a prediction model from the results of genome-wide association studies of the same race population and generation of a prediction model from the results of genome-wide association studies of different race populations. The difference is as follows. When a prediction model is generated from the results of genome-wide association studies of the same race population, a trait is predicted successfully even though the prediction model includes a single-nucleotide polymorphism having a less statistically significant effect. When a prediction model is generated from the results of genome-wide association studies of different race populations, a trait is not predicted successfully if a single-nucleotide polymorphism, which is less effective, is included in the prediction model.

There is no difference among race populations in the influence of a single-nucleotide polymorphism having a more statistically significant effect. The prediction model can thus be used for trait prediction. On the other hand, there is a difference among race populations in the influence a single-nucleotide polymorphism having a less statistically significant effect. It is thus considered that when a prediction model is generated from the results of different race populations, the inclusion of a single-nucleotide polymorphism having a less effect in the prediction model will have an adverse influence on the trait prediction.

As a result of the above observation, regarding a trait prediction model generated from genome-wide summary statistics on association between a single-nucleotide polymorphism and a trait, the present inventors generated a plurality of prediction models, such as a prediction model including only a single-nucleotide polymorphism having a large effect and a prediction model including a single-nucleotide polymorphism having a small effect, from the results of genome-wide association studies of the same race population, and simultaneously generated a plurality of prediction models from the results of genome-wide association studies of different race populations, further generated a plurality of prediction models from a summary statistic obtained by integrating a plurality of summary statistics by meta-analysis, and performed ensemble learning of these prediction models by appropriate regularized regression. They have found out that it is possible to generate a prediction model with higher prediction accuracy than the prediction models generated from the results of genome-wide association studies of the same race population and those generated from the results of genome-wide association studies of different race populations.

Hereinafter, a trait prediction model generation apparatus, a trait prediction apparatus and a trait prediction model generation method according to the embodiments will be described with reference to the drawings.

The trait prediction model generation apparatus is a computer which generates a prediction model for predicting a trait. The trait prediction apparatus is a computer which predicts the trait of an individual using a prediction model generated by the trait prediction model generation apparatus. Hereinafter, a prediction model for predicting a trait will be referred to as a trait prediction model. The trait prediction model is a mathematical model or a machine learning model that is learned to receive single-nucleotide polymorphism data of one individual and output a trait value corresponding to the trait of the one individual. In the following embodiments, a single-nucleotide polymorphism may also be referred to as a polymorphism.

### First Embodiment: Trait Prediction Model Generation Apparatus

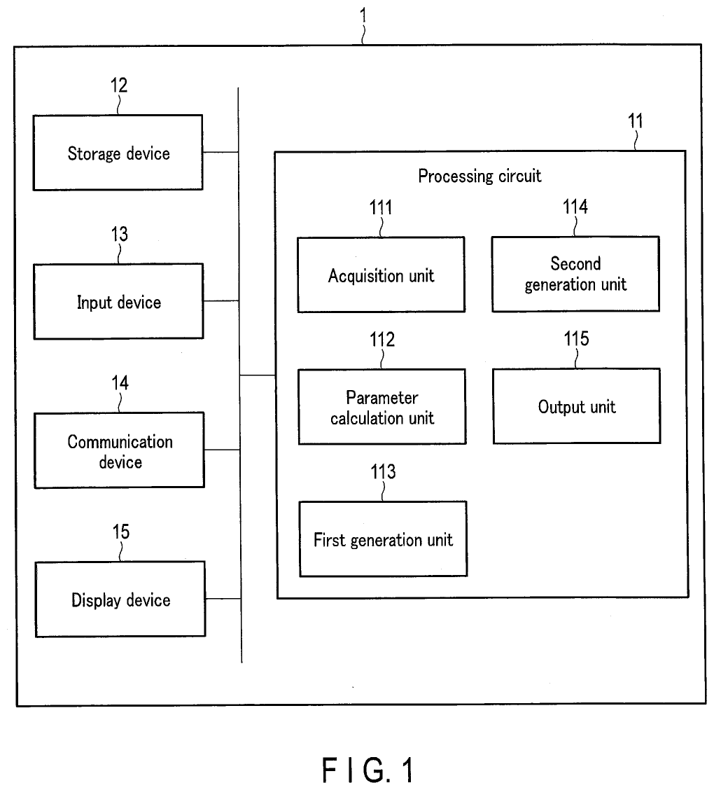

FIG. 1 is a block diagram showing an example of a configuration of a trait prediction model generation apparatus 1 according to a first embodiment. As shown in FIG. 1, the trait prediction model generation apparatus 1 includes a processing circuit 11, a storage device 12, an input device 13, a communication device 14 and a display device 15.

The processing circuit 11 includes a processor such as a central processing unit (CPU) and a memory such as a random access memory (RAM). The processing circuit 11 generates a trait prediction model. The processing circuit 11 executes programs stored in the storage device 12 to implement an acquisition unit 111, a parameter calculation unit 112, a first generation unit 113, a second generation unit 114 and/or an output unit 115. The hardware implemented on the processing circuit 11 is not limited to these units. The processing circuit 11 may be configured by, for example, an application specific integrated circuit (ASIC) to implement the acquisition unit 111, parameter calculation unit 112, first generation unit 113, second generation unit 114 and output unit 115. The acquisition unit 111, parameter calculation unit 112, first generation unit 113, second generation unit 114 and/or output unit 115 may be implemented on a single integrated circuit or individually on a plurality of integrated circuits. The functions of the acquisition unit 111, parameter calculation unit 112, first generation unit 113, second generation unit 114 and/or output unit 115 or programs for causing a computer to fulfill the functions may be recorded on a non-transitory computer-readable recording medium.

The acquisition unit 111 acquires various types of information for generating a trait prediction model. For example, the acquisition unit 111 may acquire parameters for generating a trait prediction model, such as summary statistics and inter-polymorphism correlated information. The summary statistics are parameters representing an association between a single-nucleotide polymorphism and a trait. The summary statistics are related to genome-wide association studies (GWAS) and are GWAS statistics. The summary statistics include summary statistics for one population and summary statistics for a plurality of populations. Hereinafter, the summary statistics for one population will be referred to as individual summary statistics, the summary statistics for a plurality of populations will be referred to as integrated summary statistics, and they will be referred to as summary statistics when they are not specifically distinguished from each other. The inter-polymorphism correlated information is a parameter representing a correlation among single nucleotide polymorphism. As the inter-polymorphism correlated information, a parameter representing the degree of linkage disequilibrium (LD), such as an LD reference panel, is used. The acquisition unit 111 also acquires a data set including a combination of single-nucleotide polymorphism data and its corresponding trait value. Note that the acquisition, unit 111 can also acquire the single-nucleotide polymorphism data and the trait value separately.

The parameter calculation unit 112 calculates parameters for generating a trait prediction model, such as summary statistics and inter-polymorphism correlated information. For example, the parameter calculation unit 112 conducts a meta-analysis of a plurality of individual summary statistics to calculate .integrated summary statistics.

The first generation unit 113 generates a plurality of first trait prediction models for each of the populations based on the summary statistics and the inter-polymorphism correlated information.

The second generation unit 114 generates a second trait prediction model for a specific one of the populations based on the regularized regression of the first trait prediction models for each of the populations, using a plurality of data sets including single-nucleotide polymorphism data and its corresponding trait value.

The output unit 115 outputs the second trait prediction model generated by the second generation unit 114.

The storage device 12 includes a read only memory (ROM), a hard disk drive (HDD), a solid state drive (SSD), an integrated circuit storage device, and the like. The storage device 12 stores results of various computations performed by the processing circuit 11, various programs executed by the processing circuit 11, and the like.

The input device 13 receives various commands from a user. As the input device 13, for example, a keyboard, a mouse, various switches, a touch pad, and a touch panel display can be used. The signal output from the input device 13 is supplied to the processing circuit 11. Note that the input device 13 may be a computer connected to the processing circuit 11 by wire or wirelessly.

The communication device 14 is an interface for performing information communication with an external device connected via a network.

The display device 15 displays various types of information. As the display device 15, a cathode-ray tube (CRT) display, a liquid crystal display, an organic electroluminescence (EL) display, a light-emitting diode (LED) display, a plasma display, or any other display known in the art, can be used as appropriate.

Next is a description of an example of a process of the trait prediction model generation apparatus 1.

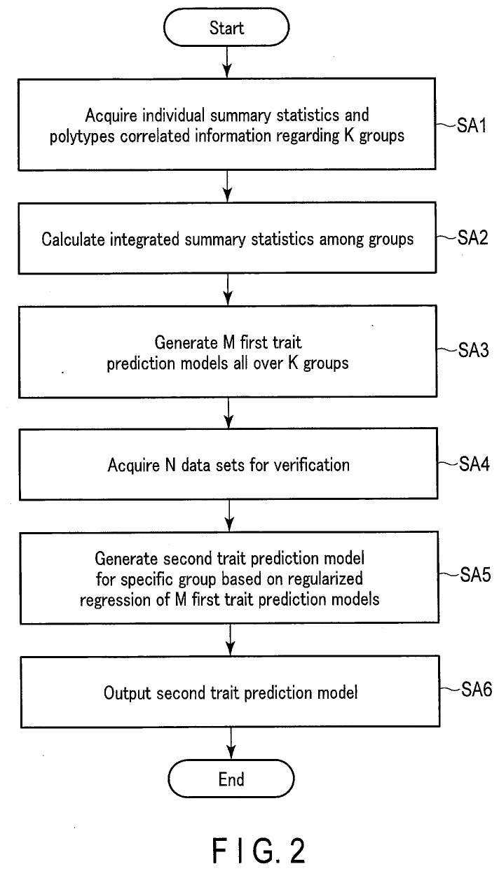

FIG. 2 is a flowchart showing an example of a process of the trait prediction model generation apparatus 1. As shown in FIG. 2, first, the acquisition unit 111 acquires individual summary statistics and inter-polymorphism correlated information concerning K (an integer of two or more) populations (step SA1). The populations may include populations of any unit, such as race populations, geographical populations, racial populations and biological populations. For the purposes of specific descriptions, however, it is assumed below that the populations are race ones. For example, population A may be Japanese, population B may Chinese and population C may be European.

The individual summary statistics and inter-polymorphism correlated information are parameters obtained from the genome-wide association studies. The individual summary statistics and inter-polymorphism correlated information are calculated based on an association (correlation) between single-nucleotide polymorphism data and its corresponding trait value.

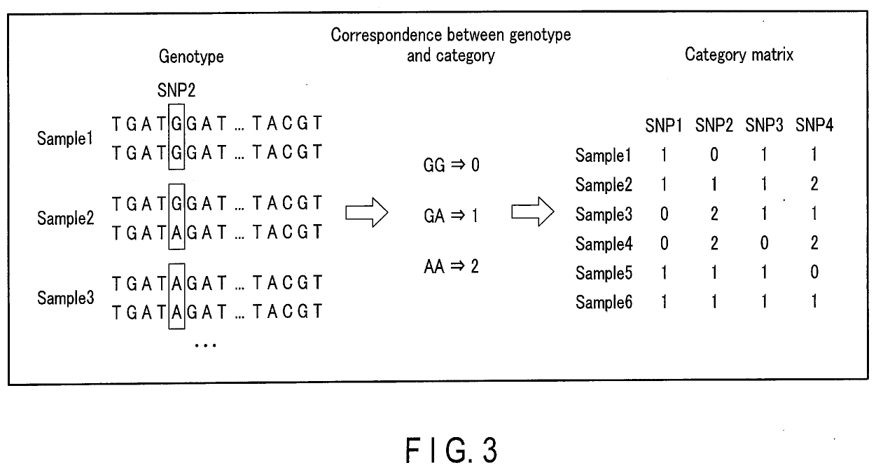

FIG. 3 is a diagram showing an example of the single-nucleotide polymorphism data. The single-nucleotide polymorphism data is data on an individual single-nucleotide polymorphisms (SNP). The single-nucleotide polymorphism data may be represented by a genotype or a category matrix.

Assume that the single-nucleotide polymorphism data represented by a genotype is, for example, serial data of bases constituting the base sequence of each individual and includes base data in at least one locus (DNA position) which can be different from a standard base sequence. The base data may be represented by a symbol such as A, T, G and C and an optional number, letter, code or the like. In the first embodiment, a single DNA position that can be different from the standard base sequence will be referred to as an SNP. Here, each of the bases in the SNP is also referred to as allele. The single-nucleotide polymorphism data represented by a genotype is acquired from an external computer or the like by the acquisition unit 111.

The single-nucleotide polymorphism data represented by a category matrix includes data of a category (classification value) indicating whether two alleles coincide with a base sequence to be a reference for at least one SNP. Assume that the reference allele in SNP2 is “G” as shown in FIG. 3, for example. In this case, the genotype of sample 1 in SNP2 is “GG” and both match the reference allele. The category of sample 1 in SNP2 is classified as “0.” Similarly, the genotype of sample 2, is “GA” and only one matches the reference allele (the other does not match the reference allele) and thus the category of sample 2 is classified as “1.” The genotype of sample 3 is “AA” and neither matches the reference allele and thus the category of sample 3 is classified as “2.” The single-nucleotide polymorphism data represented by the category matrix may be calculated based on the single-nucleotide polymorphism data represented by the genotype by the parameter calculation unit 112 or may toe acquired from an external computer or the like by the acquisition unit 111.

Here is a description of a specific method for calculating individual summary statistics by genome-wide association studies. In the following description, the single-nucleotide polymorphism data is defined as data represented by a category. The single-nucleotide polymorphism data represented by a category will also be referred to as polymorphism information.

The genome-wide association studies are a method for finding out an association (correlation) between each single-nucleotide polymorphism and a trait of interest by multiple tests. If there are p polymorphisms in all, p regression models shown in the following equation (1) is applied to a trait value y. The trait value y is the value of a trait to be predicted. If a trait to be predicted is the presence or absence of disease, the trait value y takes a binary value. If a trait to be predicted is HbAlc, the trait value y takes a continuous value.

h{E(y|Z,xⱼ)}=βₒⱼZ+βⱼxⱼ (1)

In the above equation (1), j is the number of single-nucleotide polymorphisms and takes an integer from 1 to p, Z is a covariate including an intercept term, such as age and sex, h is a link function, and xⱼ is an explanatory variable representing polymorphism information (0, 1, or 2) of the j-th SNP. The link function h is a function that connects an expected value E (y|Z, xⱼ) of the trait value y at the time of the covariate Z and the polymorphism information xⱼ to the regression model based on the covariate Z and the polymorphism information xⱼ. As the link function h, logistic regression may be used if a trait to be predicted is represented by a binary value such as the presence or absence of disease, and linear regression may be used if a trait to be predicted is represented by a continuous value such as HbAlc.

Applying p regression models to a given population, p regression coefficients β₁, . . . , βₚ and standard errors se₁, . . . , seₚ seₚ of the p regression coefficients can be calculated. The regression coefficient β and standard error se are individual summary statistics for evaluating the association of each polymorphism with a trait in this population. The regression coefficient β and standard error se are a type of GWAS statistics.

The individual summary statistics are calculated based on single-nucleotide polymorphism data and trait value for each individual in a specific population. Various types of international consortia have published summary statistics as an outcome, and these individual summary statistics may be used.

Specifically, a linkage disequilibrium coefficient r² is used as the inter-polymorphism correlated information described above. The linkage disequilibrium coefficient r² can be calculated, for example, based on polymorphism information at the individual level published as a result of the 1000 Genomes Project. More specifically, the genotype frequency and allele frequency of each SNP are calculated based on the polymorphism information, and the linkage disequilibrium coefficient r² between two SNPs is calculated based on the genotype frequency and allele frequency between the two SNPs.

The individual summary statistics and the inter-polymorphism correlated information may be acquired by the acquisition unit 111 from an external device or may be calculated by the parameter calculation unit 112 using the above-described method.

After step SA1, the parameter calculation unit 112 calculates integrated summary statistics between populations (step SA2). Meta-analysis is a method for comparing the results from the respective populations with the standardized index and integrating the results as a whole. In step SA2, the parameter calculation unit 112 performs a meta-analysis of a plurality of individual summary statistics to calculate the integrated summary statistics. For example, it performs a meta-analysis of the individual summary statistics of the Japanese population and the individual summary statistics of the European population to calculate integrated summary statistics of the Japanese and European populations. The meta-analysis method is not particularly limited, but may be any method such as a sample size method and an inverse dispersion method. Below is a description of a method for calculating the integrated summary statistics when the inverse dispersion method is used as a meta-analysis method.

If the individual summary statistics of the population k are βₖ and seₖ and wₖ is equal to 1/seₖ² for each polymorphism in order to calculate the integrated summary statistics from the summary statistics calculated for each of the K populations, the integrated summary statistics are expressed by the following equations (2).

$$β = \frac{{\sum }_{k} {β}_{k} {w}_{k}}{{\sum }_{k} {w}_{k}} , s e = \sqrt{\frac{1}{{\sum }_{k} {w}_{k}}} \qquad ( 2 )$$

The calculation of the integrated summary statistics based on the meta-analysis of the individual summary statistics may be performed using a program such as METAL.

After step SA2, the first generation unit 113 generates M (an integer of two or more) first trait prediction models all over the K populations (step SA3). Non-patent literatures 1 and 2 disclose a method for generating a trait prediction model from the summary statistics of the association between comprehensive (genome-wide) polymorphism and a trait. This method allows a first trait prediction model to be generated.

Specifically, based on the summary statistics and the inter-polymorphism correlated information, the first generation unit 113 generates a plurality of first trait prediction models using mutually different algorithms and reference values for the summary statistics and inter-polymorphism correlated information. As the algorithms, any algorithm such as PRSice2 and LDPred may be used. As a reference value for the summary statistics, for example, a threshold value for the P value is used. As a reference value for the inter-polymorphism correlated information, for example, a threshold value for the linkage disequilibrium coefficient is used. In this case, the first generation unit 113 sets a plurality of threshold values for the P value and a plurality of threshold values for the linkage disequilibrium coefficient to generate a plurality of first trait prediction models using PRSice2 for every combinations of the threshold values for the P value and the threshold values for the linkage disequilibrium coefficient and generate a plurality of first trait prediction models using LDPred for every combinations of the threshold values for the P value and the threshold values for the linkage disequilibrium coefficient. The first generation unit 113 generates a plurality of first trait prediction models for a plurality of populations using mutually different algorithms and reference values for the summary statistics and inter-polymorphism correlated information as described above. The first generation unit 113 generates a first trait prediction model of one population based on the individual summary statistics of the one population. The first generation unit 113 also generates a first trait prediction model of a plurality of populations based on the integrated summary statistics and the inter-polymorphism correlated information of the populations. Accordingly, a large number of first trait prediction models are generated in step SA3.

When it is the objective to test the association of genome-wide polymorphisms with traits (when it is the objective to test the null hypothesis that the regression coefficient βⱼ is 0), the number p of polymorphisms is usually in the range of hundreds of thousands to tens of millions. Since a number of hypothesis tests are repeated, the P value is required to satisfy a strict significance level with multiple test corrections, such as 5×10⁻⁸, in order to control a false positive. The P value is calculated from Φ⁻¹(−2|β/se|) using the inverse function Φ⁻¹ of the cumulative frequency function of the normal distribution.

On the other hand, when it is the objective to make a prediction, the P value does not satisfy the significance level but may include a polymorphism that is useful for the prediction. Thus, a larger P value of, e.g., 1×10⁻² is vised. The P value is selected depending on the performance of a trait prediction model and varies depending on traits such as a genetic structure. Thus, the P value needs to be selected appropriately.

In the method of non-patent literature 1, the relationship between single-nucleotide polymorphisms and trait values is estimated by a linear regression model by assuming the independence between the single-nucleotide polymorphisms and using the regression coefficient of the summary statistics. The assumption of the independence between the single-nucleotide polymorphisms does not hold true for single-nucleotide polymorphisms having a linkage disequilibrium relation. Thus, single-nucleotide polymorphisms having a linkage disequilibrium relation are selected in advance by the threshold value of a predetermined linkage disequilibrium coefficient r², and only the population A of single-nucleotide polymorphisms of the selected single-nucleotide polymorphisms, in which the P value is not larger than a predetermined threshold value, is used. The index of the single-nucleotide polymorphisms (SNP) included in the population A is represented by j as described above. In this case, a predicted value PRSₘ output from the first trait prediction model is calculated based on the polymorphisms information xⱼ of the j-th SNP and the regression coefficient βⱼ according to the following equation (3). Since the single-nucleotide polymorphism used to calculate the predicted value PRSₘ varies depending on the combination of the threshold value of the linkage disequilibrium coefficient r² and the threshold value of the P value, the prediction accuracy also varies depending on the combination.

$${PRS}_{m} = {\sum }_{j ∈ A} {β}_{j} {x}_{j} \qquad ( 3 )$$

In the foregoing description, the first generation unit 113 generates a plurality of first trait prediction models by changing both the reference value for the summary statistics and the reference value for the inter-polymorphism correlated information. However, it may generate a plurality of first trait prediction models by changing only one of the reference values.

After step SA3, the acquisition unit 111 acquires N (N is an integer of one or more) validation data sets (step SA4). The validation data sets are data sets of persons belonging to a specific population to be predicted by the second trait prediction model.

After step SA4, the second generation unit 114 generates a second trait prediction model for a specific population based on regularized regression of M first trait prediction models (step SA5). The second trait prediction model is generated by ensemble learning of the M first trait prediction models.

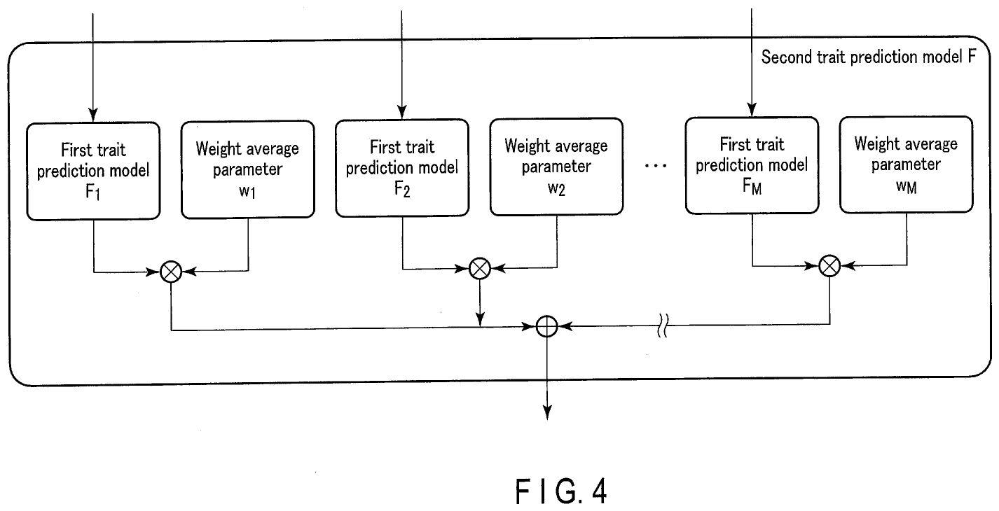

FIG. 4 is a diagram showing an example of a configuration of a second trait prediction model P. As shown in FIG. 4, a plurality of first trait prediction models Fₘ are generated in step SA3. The first trait prediction model Fₘ receives single-nucleotide data of an individual i and outputs a first trait value PRS_{i,m} of the individual. The second trait prediction model F is configured to calculate the sum total PRS of the product of the output value PRS_{i,m} of each the first trait prediction models Fₘ and the weight parameter (hereinafter referred to as weighted average parameter) wₘ for the first trait prediction model Fₘ over the first trait prediction models Fₘ of each of the populations. That is, based on the first trait value PRS_{i,m} output from the first trait prediction model Fₘ and the weighted average parameter wₘ corresponding to the first trait prediction model Fₘ, the second trait value PRSᵢ, which is the output value of the second trait prediction model F, is calculated according to the following equation (4).

$$P R {S}_{i} = {\sum }_{m ∈ M} {w}_{m} {PRS}_{i , m} \qquad ( 4 )$$

The calculation of the second trait prediction model F results in the calculation of a set w\hat{ } of a plurality of weighted average parameters wₘ corresponding to their respective first trait prediction models Fₘ. The second generation unit 114 calculates a weighted average parameter based on the regularized regression of the first trait prediction models Fₘ using N validation data sets. Specifically, based on the N validation data sets, the second generation unit 114 determines a value of the weighted average parameter wₘ to minimize an objective function including a loss function between a predicted value and a trait value and a regularization term for the weighted average parameter wₘ. The regularized regression may employ any method such as Ridge regression, Lasso regression and Elastic Net regression. The Ridge regression includes L2 regularization as a regularization term. The Lasso regression includes an L1 regularization term as a regularization term. The Elastic Net regression includes the sum of the L1 regularization term and the L2 regularization term as a regularization term.

When Elastic Net regression is used, the minimization of the objective function of the weighted average parameter set w\hat{ } is expressed by the following equation (5).

ŵ=argmin_{w}{Σᵢ₌₁^{N}(yᵢ−PRSᵢ)²−λΣⱼ₌₁^{M}(α|wⱼ|+(1−α)wⱼ²)} (5)

In the equation (5), λ and α are hyperparameters of the Elastic Net regression, and, more specifically, λ represents regularization strength and a represents a parameter that balances a penalty for the L1 regularization term and a penalty for the L2 regularization term.

The second generation unit 114 determines a weighted average parameter set w\hat{ } and hyperparameters λ and α by k-fold cross-validation using a validation data set. Specifically, the second generation unit 114 divides the N validation data sets acquired in step SA4 into k data sets, applies k−1 validation data sets to an objective function under any hyperparameters λ and α to determine a weighted average parameter set w\hat{ }, and applies the remaining one validation data set to the second trait prediction model F under the determined weighted average parameter set w\hat{ } to calculate an output value PRS and thus calculate prediction accuracy based on the output value PRS. The remaining one validation data set may also be referred to as an evaluation data set.

The second generation unit 114 repeats the determination of the weighted average parameter set w\hat{ } and the calculation of the prediction accuracy k times so that all of the k validation data sets each become an evaluation data set. After the k repetitions, the second generation unit 114 determines optimum hyperparameters λ and α to maximize the prediction accuracy, sets the hyperparameters λ and α to an objective function, determines a final weighted average parameter set w\hat{ } using the objective function, and sets the determined weighted average parameter set w\hat{ } to a weighted average parameter set w\hat{ } regarding a specific race population. Accordingly, a second trait prediction model for a specific race population is generated by modeling ensemble learning of a plurality of first trait prediction models Fₘ.

Note that the method for determining the weighted average parameter set w\hat{ } and the hyperparameters λ and α is not limited to the above but can be changed as appropriate. For example, the number of repetitions of the determination of the weighted average parameter set w\hat{ } and the calculation of the prediction accuracy is not limited to k, but may be smaller or larger than k.

The second generation unit 114 can generate a second trait prediction model for a race population to be generated by executing step SA5 using the validation data set for the race population.

After step SA5, the output unit 115 outputs the second trait prediction model generated in step SA5 (step SA6). In step SA6, the output unit 115 stores the second trait prediction model in the storage device 12 and transmits it to the trait prediction device 2. Specifically, the second trait prediction model is data of the combination of a plurality of first prediction models and a plurality of weighted average parameter sets. The second trait prediction model is managed in association with an identifier representing a corresponding race type.

When step SA6 is executed, the operation of the trait prediction model generation apparatus 1 is terminated.

### EXAMPLE

Next is a description of an example of the trait prediction model generation apparatus 1 according to the first embodiment. In this example, a trait prediction model according to the first embodiment is generated and evaluated by focusing on the disease of type 2 diabetes as an example of a multifactorial qualitative trait. As the summary statistics for a correlation between a single-nucleotide polymorphism and the disease of type 2 diabetes, the summary statistics for East Asians published by the Asian Genetic Epidemiology Network and those for Europeans published by the DIAGRAM Consortium were used. The correlation matrix between single-nucleotide polymorphisms was calculated using polymorphism information at the individual level published by the 1000 Genomes Project. For the validation data set and the evaluation data set, 8,444 persons of the Tohoku Medical Megabank Project were used, and ⅔ of the 8,444 persons were used for the validation data set and ⅓ thereof were used for the evaluation data set.

The following four different trait prediction models were confirmed: (1) a trait prediction model with the highest prediction accuracy in a validation data set among the prediction models generated using PRSice2 only from the summary statistics of East Asians; (2) a trait prediction model with the highest prediction accuracy in a validation data set among the prediction models generated using LDPred only from the summary statistics of East Asians; (3) a trait prediction model with the highest prediction accuracy in a validation data set using Elastic Net regression among a plurality of trait prediction models generated using PRSice2 and LDPred from the summary statistics of East Asians; and (4) a trait prediction model with the highest prediction accuracy in a validation data set using Elastic Net regression among a plurality of trait prediction models generated using PRSice2 and LDPred from the summary statistics of East Asians, these of Europeans, and those obtained by performing a meta-analysis of the summary statistics of East Asians and Europeans. The fourth trait prediction model corresponds to the second trait prediction model according to the first embodiment.

36 trait prediction models were generated using PRSice2 as follow. For single-nucleotide polymorphism data at the individual level to calculate a correlation between polymorphisms, samples of the same race population are extracted from the 1000 Genomes Project and used. For reference values (threshold values) for linkage disequilibrium coefficients, 0.2, 0.4, 0.6 and 0.3 are set. For reference values (threshold values) for P values, 5×10⁻⁸, 1×10⁻⁷, 1×10⁻⁶, 1×10⁻⁵, 1×10⁻⁴, 1×10⁻, 1×10⁻², 1×10⁻¹ and 1 are set. For the other parameters, default values of PRSice2 are set. The generated 36 trait prediction models correspond to the number of combinations between the threshold values of the linkage disequilibrium coefficients and those of the P values.

7 trait prediction models are generated using LDPred as follows. For the single-nucleotide polymorphism data, samples of the same race population were extracted from the 1000 Genomes Project and used. For the ρ parameter that is a set parameter of LDPred, the default values of 1.3×10⁻¹, 1×10⁻¹, 3×10⁻², 1×10⁻², 3×10⁻³ and 1×10⁻³ are used.

The trait prediction model (4) is generated as a second trait prediction model for Japanese by generating 36 first trait prediction models using PRSice2 and 7 first trait prediction models using LDPred for each of Fast Asians and Europeans and performing ensemble learning of the 36 first trait prediction models and 7 first trait prediction models for East Asians and the 36 first trait prediction models and 7 first trait prediction models for Europeans, based on the validation data set for Japanese.

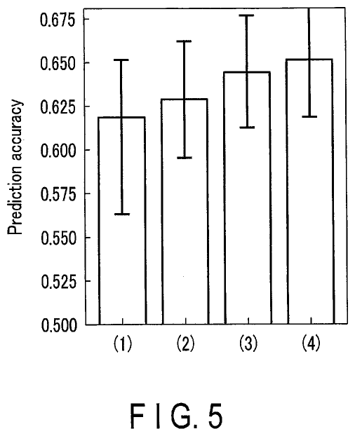

FIG. 5 is a bar chart showing the prediction accuracy of the above four different trait prediction models. As shown in FIG. 5, the prediction accuracy of the trait prediction model (1) by AUC in the validation data set is 61.8%, the prediction accuracy of the trait prediction model (2) is 62.3%, the prediction accuracy of the trait prediction model (3) is 64.4%, and the prediction accuracy of the trait prediction model (4) (the second trait prediction model, according to the first embodiment) is 65.1%. The prediction accuracy of the second trait prediction model according to the first embodiment is the highest.

As described above, the trait prediction model generation apparatus 1 according to the first embodiment includes the first generation unit 113 and second generation unit 114. The first generation unit 113 generates a plurality of first trait prediction models Fₘ based on the summary statistics and the inter-polymorphism correlated information for each of the populations. The second generation, unit 114 generates a second trait prediction model F for a specific one of the populations based on the regularized regression of the first trait prediction models Fₘ using a plurality of data sets including single-nucleotide polymorphism data and trait values.

As described above, the second trait prediction model F is generated by modeling ensemble learning of the first trait prediction models Fₘ. Factors having a strong effect have only to be averaged because they have the same effect irrespective of a difference in the populations, and factors having a weak effect have to obtain information from the same population because they are not used for prediction unless they belong to the same population. The ensemble learning allows the factors having strong and weak effects to be learned optimally for a specific population. It is thus possible to generate a second trait prediction model F that is optimum for the specific population.

According to the first embodiment, therefore, a polygenic model with high prediction accuracy can be generated.

### First Embodiment: Trait Prediction Apparatus

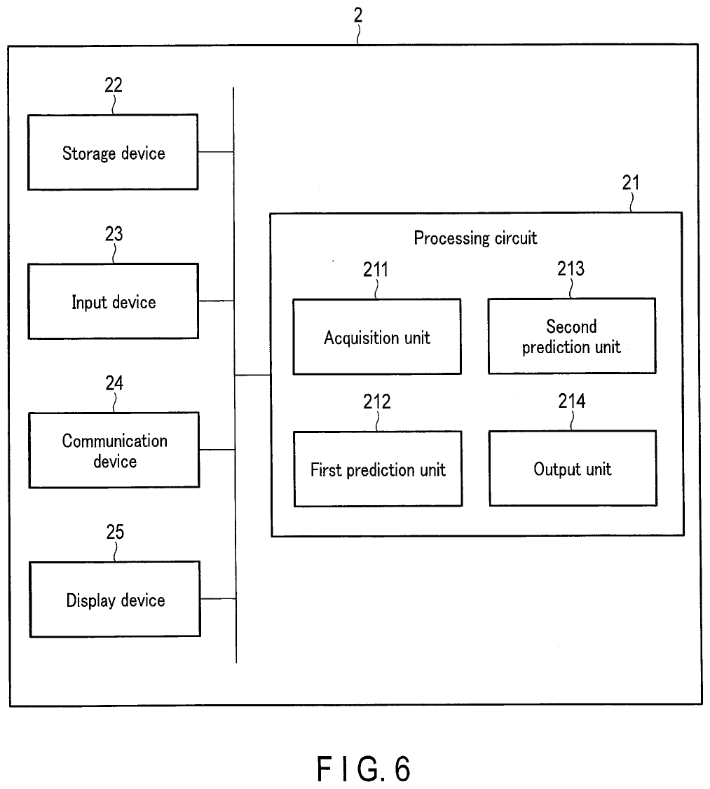

FIG. 6 is a block diagram showing an example of a configuration of a trait prediction apparatus 2 according to the first embodiment. As shown in FIG. 6, the trait prediction apparatus 2 includes a processing circuit 21, a storage device 22, an input device 23, a communication device 24 and a display device 25.

The processing circuit 21 includes a CPU and a memory such as a RAM. The processing circuit 21 predicts a trait of an individual using a second trait prediction model. The processing circuit 21 executes programs stored in the storage device 22 to implement an acquisition unit 211, a first prediction unit 212, a second prediction unit 213 and/or an output unit 214. The hardware implemented on the processing circuit 21 is not limited to these units. The processing circuit 21 may be configured by, for example, an application specific integrated circuit (ASIC) to implement the acquisition unit 211, first prediction unit 212, second prediction unit 213 and/or output unit 214. The acquisition unit 111, first, prediction unit 212, second prediction unit 213 and/or output unit 214 may be implemented on a single integrated circuit or individually on a plurality of integrated circuits. The functions of the acquisition unit 211, first prediction unit 212, second prediction unit 213 and/or output unit 214 or programs for causing a computer to fulfill the functions may be recorded on a non-transitory computer-readable recording medium.

The acquisition unit 211 acquires various types of information. For example, the acquisition unit 211 acquires single-nucleotide polymorphism data or the like of an individual whose trait is to be predicted. The acquisition unit 111 may also acquire the second trait prediction model generated by the trait prediction model generation apparatus 1. Specifically, the acquisition unit 211 acquires a plurality of first trait prediction models and a plurality of weighted average parameters corresponding to the first trait prediction models, as the second trait prediction model.

The first prediction unit 212 applies single-nucleotide polymorphism data of one individual to the first trait prediction models to calculate a plurality of first trait values for the one individual.

The second prediction unit 213 calculates a second trait value for one individual on the basis of the first trait values calculated by the first prediction unit 212 and the weighted average parameters which are associated with a population to which the individual belongs and which correspond to their respective first trait prediction models.

The output unit 214 outputs the second trait value calculated by the second prediction unit 213.

The storage device 22 includes a ROM, an HDD, an SSD, an integrated circuit storage device, and the like. The storage device 22 stores results of various computations performed by the processing circuit 11, various programs executed by the processing circuit 11, and the like. The storage device 22 also stores the second trait prediction model generated by the trait prediction model generation apparatus 1 in association with an identifier representing a race type. Specifically, the storage device 22 stores a plurality of first trait prediction models and a plurality of weighted average parameters corresponding to the first trait prediction models, as the second trait prediction model.

The input device 23 receives various commands from a user. As the input, device 23, for example, a keyboard, a mouse, various switches, a touch pad, and a touch panel display can be used. The signal output from the input device 23 is supplied to the processing circuit 21. Note that the input device 23 may be a computer connected to the processing circuit 21 by wire or wirelessly.

The communication device 24 is an interface for performing information communication with an external device connected via a network.

The display device 25 displays various types of information. As the display device 25, a CRT display, a liquid crystal display, an organic EL display, an LED display, a plasma display, or any other display known in the art, can be used as appropriate.

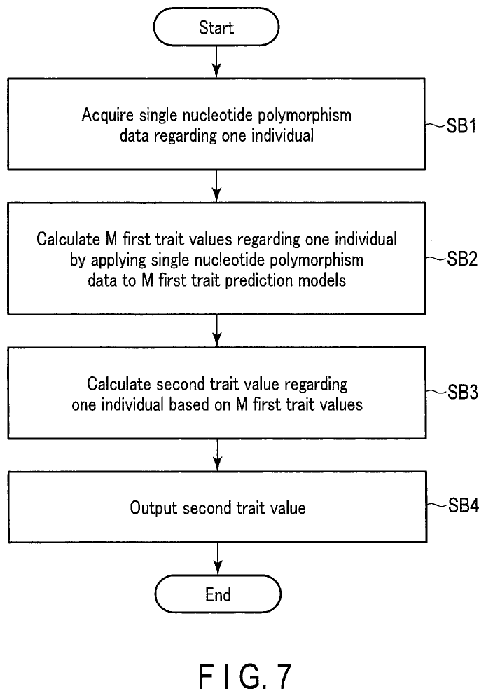

Next is a description of an example of a process of the trait prediction apparatus 2 according to the first embodiment. FIG. 7 is a flowchart of the example of a process of the trait prediction apparatus 2. As shown in FIG. 7, first, the acquisition unit 211 acquires single-nucleotide polymorphism data for one individual whose trait is to be predicted (step SB1).

After step SB1, the first prediction unit 212 applies the single-nucleotide polymorphism data acquired in step SB1 to M first trait prediction models to calculate M first trait values for the one individual whose trait is to be predicted (step SB2). After step SB2, the second prediction unit 213 calculates a second trait value for the one individual whose trait is to be predicted, based on the M first trait values calculated in step SB2 (step SB3). After step SB3, the output unit 214 outputs the second trait value calculated in step SB3 (step SB4). In step SB4, for example, the output unit 214 may display the second trait value on the display device 25, record it in the storage device 22, or transmit it to another computer via the communication device 24.

When step SB4 is executed, the operation of the trait prediction apparatus 2 is terminated.

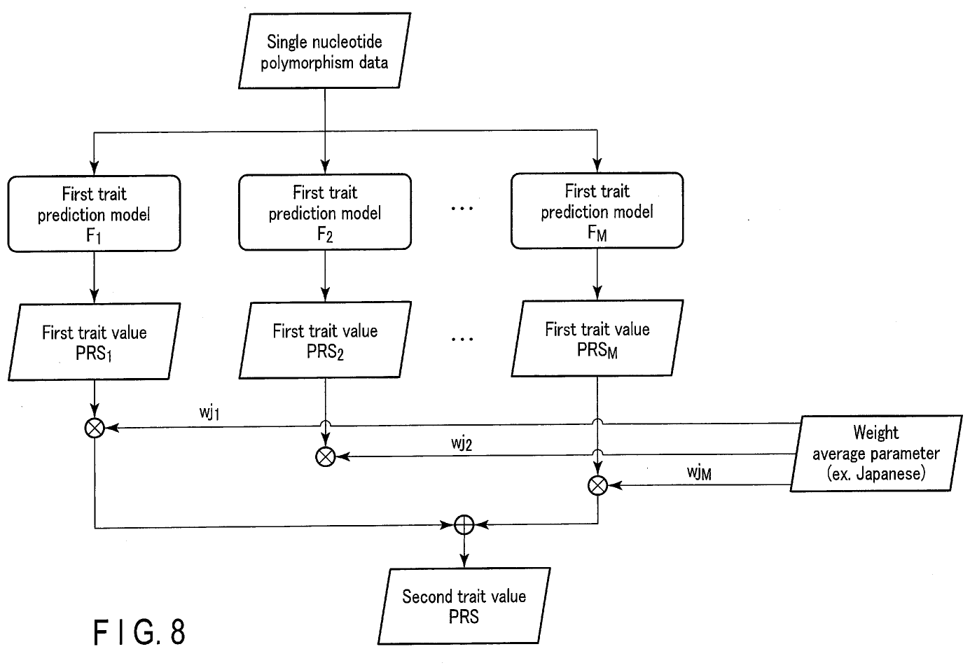

FIG. 8 is a schematic diagram of the example of a process of the trait prediction apparatus 2 shown in FIG. 7. Assume that an individual whose trait is to be predicted is, for example, a Japanese. In this case, the first prediction unit 212 reads a second trait prediction model for the Japanese from the storage device 22. Specifically, the first prediction unit 212 selects and reads a second trait prediction model associated with an identifier corresponding to the Japanese from a plurality of second trait prediction models stored in the storage device 22. As the second trait prediction model corresponding to the Japanese, M first trait prediction models Fₘ and M weighted average parameters wₘ are read out.

Then, the first prediction unit 212 applies the single-nucleotide polymorphism data for one individual whose trait is to be predicted to each of the M first trait prediction models Fₘ to calculate M first trait values PRSₘ. In accordance with the following equation (6), the second prediction unit 213 multiplies the M first trait values PRSₘ by the M weighted average parameters wₘ to calculate M integrated values, and adds the M integrated values to calculate a second trait value PRS. It is thus possible to obtain a high-accuracy second trait value PRS for one Japanese.

$$PRS = {\sum }_{m ∈ M} {w}_{m} {PRS}_{m} \qquad ( 6 )$$

As described above, the trait prediction apparatus 2 according to the first, embodiment includes the acquisition unit 211, first prediction unit 212, second prediction unit 213 and output unit 214. The acquisition unit 211 acquires single-nucleotide polymorphism data on one individual. The first prediction unit 212 applies the single-nucleotide polymorphism data to each of the first trait prediction models Fₘ to calculate a plurality of first trait values PRSₘ for one individual. The second prediction unit 213 calculates a second trait value PRS for one individual on the basis of the first trait values PRSₘ and a plurality of weighted average parameters wₘ corresponding to their respective first trait prediction models Fₘ associated with a population to which the individual belongs. The output unit 214 outputs the second trait value PRS.

As described above, the trait prediction apparatus 2 can calculate a second trait value PRS with high prediction accuracy by performing ensemble learning of the first trait prediction models Fₘ.

### Second Embodiment: Trait Prediction Model Generation Apparatus

Next is a description of a trait prediction model generation apparatus 1 according to a second embodiment. In the following description, components of the first and second embodiments, which have substantially the same function, are denoted by the same reference numeral and their overlapping descriptions will be described only when necessary.

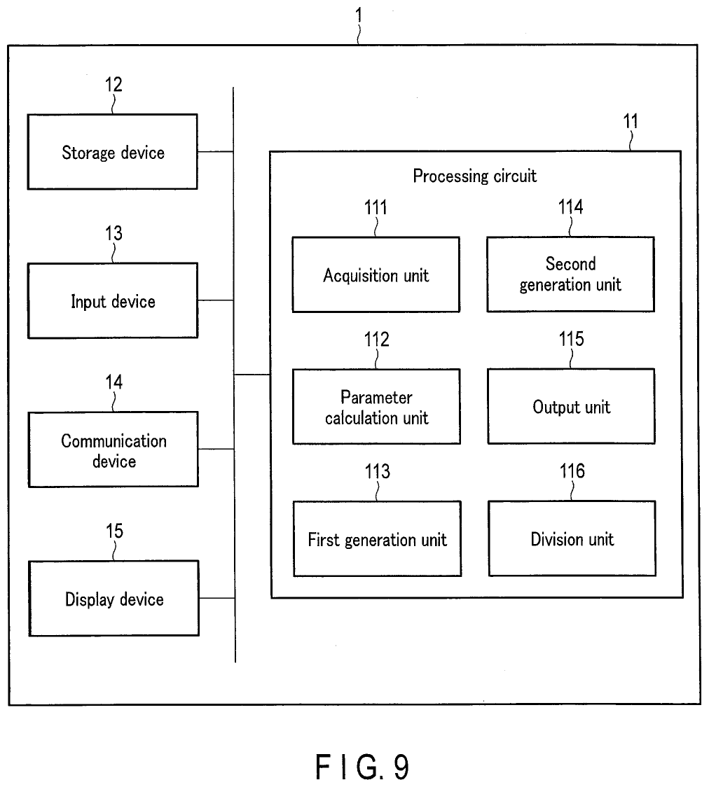

FIG. 9 is a block diagram showing an example of a configuration of the trait prediction model generation apparatus 1 according to the second embodiment. As shown in FIG. 9, the trait prediction model generation apparatus 1 according to the second embodiment includes a processing circuit 11, a storage device 12, an input device 13, a communication device 14 and a display device 15. The processing circuit 11 executes programs stored in the storage device 12 to implement an acquisition unit 111, a parameter calculation unit 112, a first generation unit 113, a second generation unit 114, an output unit 115 and/or a division unit 116. The hardware implemented on the processing circuit 11 is not limited to these units. The processing circuit 11 may be configured by, for example, an application specific integrated circuit (ASIC) to implement the acquisition unit 111, parameter calculation unit 112, first generation unit 113, second generation unit 114, output unit 115 and/or the division unit 116. The acquisition unit 111, parameter calculation unit 112, first generation unit 113, second generation unit 114, output unit 115 and/or division unit 116 may be implemented on a single integrated circuit or individually on a plurality of integrated circuits. The functions of the acquisition unit 111, parameter calculation unit 112, first generation unit 113, second generation unit 114, output unit 115 and/or division unit 116 or programs for causing a computer to fulfill the functions may be recorded on a non-transitory computer-readable recording medium.

The division unit 116 divides a single genome region into a plurality of genome regions in accordance with a correlation between single-nucleotide polymorphisms on the basis of a plurality of single-nucleotide polymorphism data for a plurality of populations. The locus (DNA location) of each of the genomic regions does not vary depending on the type of a population, but is common to a plurality of populations.

The first generation unit 113 generates a plurality of first trait prediction models of each of the populations for each of the genome regions.

The second generation unit 114 generates a second trait prediction model based on the first trait prediction models of each of the populations generated for each of the genome regions.

Next is a description of an example of a process of the trait prediction model generation apparatus 1 according to the second embodiment.

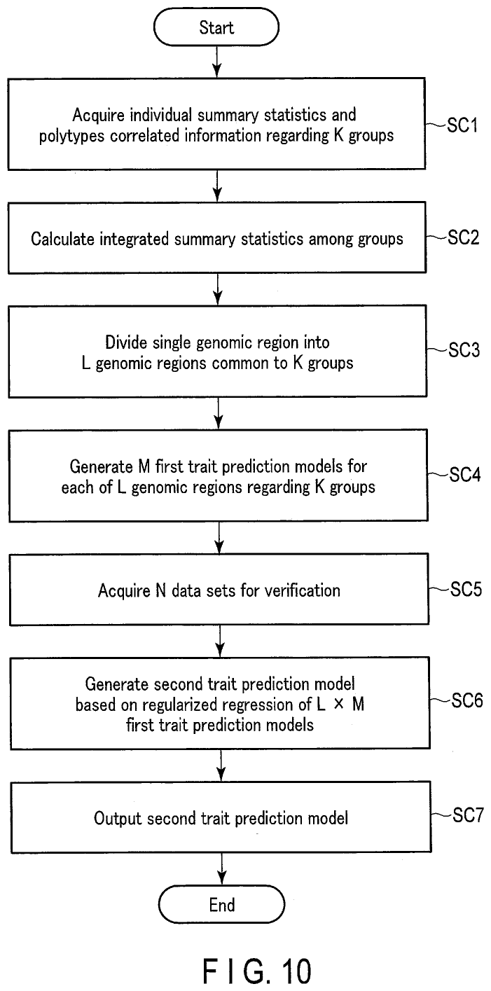

FIG. 10 is a flowchart showing an example of a process of the trait prediction model generation apparatus 1 according to the second embodiment. As shown in FIG. 10, first, the acquisition unit 111 acquires individual summary statistics and inter-polymorphism correlated information concerning K (an integer of two or more) populations (step SC1). The process in step SC1 is similar to the process in step SA2 shown in FIG. 2.

After step SC1, the parameter calculation unit 112 calculates integrated summary statistics between populations (step SC2). The process in step SC2 is similar to the process in step SA2 shown in FIG. 2.

After step SC2, the dividing unit 116 divides a genome region into L genome regions common to K populations (step SC3). A process of dividing a genome region will be described below in detail.

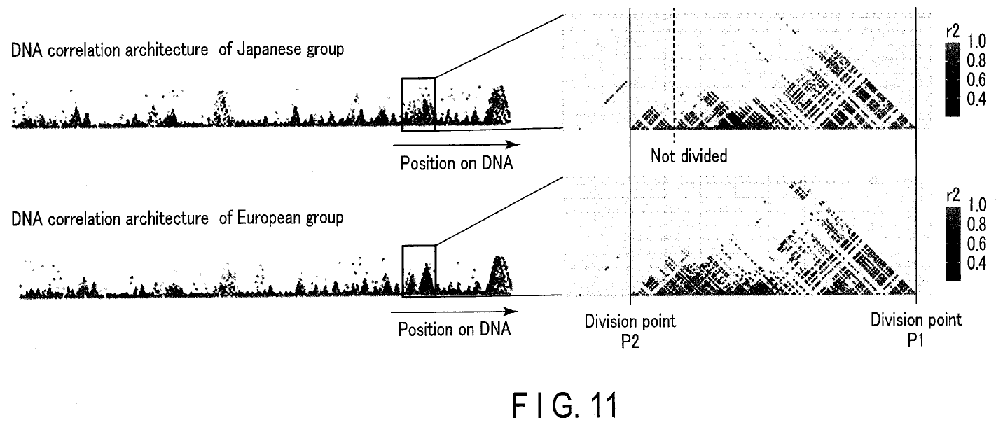

FIG. 11 a schematic diagram showing a process of dividing a genomic region in step SC3. The upper part of FIG. 11 shows an LD plot representing a correlation structure of DNA of the Japanese population, and the lower part thereof shows an LD plot representing a correlation structure of DNA of the European population. The region of interest of the LD plot shown on the left, side of each of the upper and lower parts of FIG. 11 is enlarged on the right side thereof. The points of the LD plot are assigned linkage disequilibrium coefficients r² between their corresponding SNPs. SNPs, which are physically close to each other, are strongly correlated with each other. In a specific region, SNPs, which are distant from each other, may be strongly correlated with each other. This region having strongly correlated SNPs is called an LD block. For example, among the points of the LD plot, a set of spatially continuous points having linkage disequilibrium coefficients r² is set in the LD block. The location of the LD block varies from race to race. A plurality of LD blocks are set for a base sequence.

The division unit 116 sets a plurality of LD blocks in a plurality of genome regions, respectively. Each of the genomic regions is defined as a region between the top and bottom ends P1 and P2 of the DMA location occupied by each of the LD blocks. The top and bottom ends PI and P2 define each of the genome regions. The top and bottom ends PI and P2 are also called dividing points. The division unit 116 records the combination of the positions of a division point P1 on the top side and a division point P2 on the bottom side for each of the genome regions. In this case, the division unit 116 sets a common genome region to different races. For example, as shown in FIG. 11, even though the Japanese and the Europeans are different in DNA location for the same LD block, a genome region is set to a DNA position common to Japanese and Europeans. The genome region may be set to include the LD blocks of Japanese and Europeans, or may be set to a larger or smaller one of the LD blocks thereof, or may be set to, for example, a sum region and a product region of the LD blocks thereof. The combination of locations of the division point on the top end side and the division point on the bottom end side of each genome region is stored in the storage device 12 and transmitted to the trait prediction apparatus 2.

The genome region dividing process is conceptually as described above, and an example of the algorithm will be described below. In addition, the division unit 116 constructs a genome matrix X based on M pieces of polymorphism information of N persons. However, the polymorphism information is normalized so as to be average “0” and variance “1” for each column of the matrix. The genome matrix X is an N×M dimensional matrix in which an element xᵢⱼ in the i-th row and j-th column is the j-th polymorphism information of the i-th person. In this case, the correlation between the polymorphisms is represented by a symmetric matrix of M×M dimensions of V=X^{T}X/N, and the element in the i-th row and j-th column of V is a value Indicating a correlation between the i-th polymorphism and the j-th polymorphism in a population of N persons. By approximating V as a small-dimensional symmetric matrix, such that all diagonal elements are “1,” and as a matrix, such that the other elements are “0”, it can be divided into regions having no correlation between single-nucleotide polymorphisms in the population.

In order to divide a genome region into regions having no correlation between polymorphisms common to a plurality of populations, the division unit 116 calculates matrix Vₜᵣₐₙₛ based on correlation Vₖ₁ calculated in a first population and correlation Vₖ₂ calculated in a second population in accordance with the following equation. The element of the i-th row and j-th column in the matrix Vₜᵣₐₙₛ has a correlation V_{k1, i, j} when the absolute value of the correlation V_{k1, i, j} is larger than that of a correlation V_{k2, i, j}, and it has the correlation V_{k2, i, j} when the absolute value of the correlation V_{k1, i, j} is larger than that of the correlation V_{k1, i, j}.

$${V}_{trans , i , j} = \{ \begin{matrix}{V}_{k 1 , i , j} if \vert {V}_{k 1 , i , j} \vert > \vert {V}_{k 2 , i , j} \vert \\ {V}_{k 2 , i , j} if \vert {V}_{k 2 , i , j} \vert > \vert {V}_{k 1 , i , j} \vert\end{matrix} \qquad ( 7 )$$

The division unit 116 calculates the sum of diagonal components of Vₜᵣₐₙₛ expressed by the following equation (8) and uses a point at which the sum is smaller than a reference value as a division point to divide a genome region into regions common to a plurality of populations and having no correlation between polymorphisms.

diagonal component=Σᵢ₌₁^{k}V_{trans,i,k-i+1}, (k=1,2, . . . , 2n−1) (8)

For example, the correlation calculated using Japanese in the upper part of FIG. 11 corresponds to Vₖ₁, and the correlation calculated using Europeans in the lower part thereof corresponds to Vₖ₂. The matrix Vₜᵣₐₙₛ corresponds to the larger one of Vₖ₁ and Vₖ₂ selected for each point of the LD plot. The division unit 116 draws a vertical line at an optional DNA location of the LD blot, calculates the sum of the points on the vertical line, and compares the sum with a threshold value. The threshold value may be set to an optional value. The dividing unit 116 calculates the sum while shifting the location to both right and left sides based on a DNA location where the sum exceeds a threshold value, and specifies DNA locations where the sum is smaller than the threshold value as the division points P1 and P2. In the DNA location corresponding to the legend “Not divided” in FIG. 11, the sum of Japanese is less than the threshold value, and the sum of Europeans is equal to or greater than the threshold value and thus a genome region is not divided.

When a genome region is divided into regions common to a plurality of populations and having no correlation between polymorphisms, polymorphism information of a specific race population may be used. In addition, a genome region may be divided into regions having no correlation between polymorphisms using generally available LDetect.

After step SC3, the first generation unit 113 generates M first trait prediction models for each of L genome regions for K populations (step SC4). In step SC4, the first generation unit 113 generates L×M first trait prediction models for each of the genome region using individual summary statistics and integrated summary statistics. The first trait prediction model generating method has only to be similar to that of the first embodiment.

After step SC4, the acquisition unit 111 acquires N validation data sets belonging to a race population to be generated (step SC5).

After step SC5, the second generation unit 114 generates a second trait prediction model for a specific population based on regularized regression of L×M first trait prediction models (step SC6). The second trait prediction model F of the second embodiment is configured to calculate the total sum PRSᵢ of the products of output values PRS_{i, ml} of the first trait prediction models Fₘₗ and weighted average parameter wₘₗ for the first trait prediction model Fₘₗ over the first trait prediction models Fₘₗ and the genome regions G1 of each of the populations. That is, based on the predicted value PRS_{i, ml} for the individual i and the weighted average parameter wₘₗ for the predicted value PRS_{i, ml}, which are output from the first trait prediction model Fₘₗ, the output value PRSᵢ of the second trait prediction model F is calculated in accordance with the following equation (9).

$$PR {S}_{i} = {\sum }_{m ∈ M} {\sum }_{l ∈ L} {w}_{{m}_{l}} {PRS}_{i , {m}_{l}} \qquad ( 9 )$$

The calculation of the second trait prediction model F has only to be similar to that of the second trait prediction model of the first embodiment. That is, the second generation unit 114 calculates a weighted average parameter based on the regularized regression of a plurality of first trait prediction models Fₘₗ using N data sets for validation. Specifically, based on the N data sets for validation, the second generation unit 114 determines the value of the weighted average parameter for minimizing an objective function including a loss function between a predicted value and a trait value and a regularization term for the weighted average parameter. The regularized regression may include any method such as Ridge regression. Lasso regression and Elastic Net regression.

When the Elastic Net regression is used, the minimization of the objective function of a weighted average parameter set w\hat{ } is expressed by the following equation (10). Like in the first embodiment, the second generation unit 114 can perform, for example, k-fold cross-validation using a data set for validation to determine the weighted average parameter set w\hat{ } and the hyperparameters λ and α which are optimum for maximizing the prediction accuracy.

ŵ=argmin_{w}{Σᵢ₌₁^{N}(yᵢ−PRSᵢ)²−λΣₘ₌₁^{M}Σₗ₌₁^{L}(α|wₘ_{ₗ}|+(1−α)wₘ_{ₗ}²)} (10)

The second generation unit 114 can perform the process of step SC6 using a data set for validation regarding a race population to be generated to generate a second trait prediction model for the race population.

After step SC6, the output unit 115 outputs a second trait prediction model generated in step SC6 (step SC7). In step SC7, the output unit 115 stores the second trait prediction model in the storage device 12 and transmits it to the trait prediction apparatus 2. Specifically, the second trait prediction model is data of the combination of a plurality of first prediction models and a plurality of weighted average parameter sets for each of the genome regions. The second trait prediction model is managed in association with an identifier representing its corresponding race type.

As described above, the trait prediction model generation apparatus 1 according to the second embodiment generates a first trait prediction model F_{m, l} for each of the genome regions, and generates a second trait prediction model F by modeling ensemble learning of the first trait prediction models F_{m, l} all over the gate regions. When there are genome regions which have similar effects beyond population differences and genome regions which do not have them, each of the first trait prediction models F_{m, l} can learn the properties of the genome regions individually. Since the second trait prediction model F is generated by modeling the ensemble learning of the first trait prediction models F_{m, l}, the difference in properties among the genome regions can optimally be learned for a specific population. It is thus possible to generate a second trait prediction model F that is optimum for a specific population. The second embodiment thus makes it possible to generate a polygenic model with high prediction accuracy.

### Second Embodiment: Trait Prediction Apparatus

Next is a description of a trait prediction apparatus 2 according to the second embodiment. In the following description, components of the first and second embodiments, which have substantially the same function, are denoted by the same reference numeral and their overlapping descriptions will be described only when necessary.

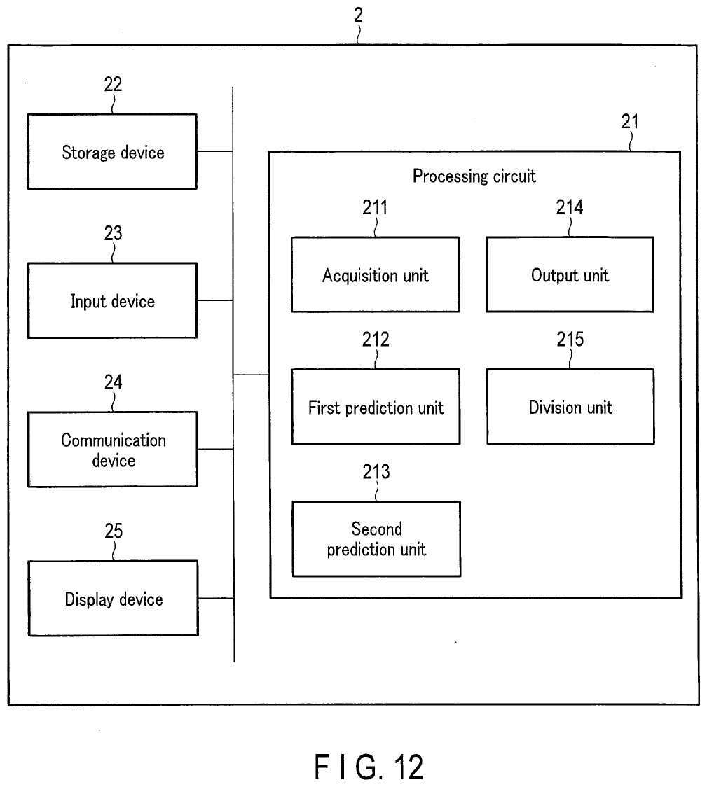

FIG. 12 is a block diagram showing an example of a configuration of the trait prediction apparatus 2 according to the second embodiment. As shown in FIG. 12, the trait prediction apparatus 2 includes a processing circuit 21, a storage device 22, an input device 23, a communication device 24 and a display device 25. The processing circuit 21 executes programs stored in the storage device 22 to implement an acquisition unit 211, a first prediction unit 212, a second prediction unit 213, an output unit 214 and/or a division unit 215. The hardware implemented on the processing circuit 21 is not limited to these units. The processing circuit 21 may be configured by, for example, an application specific integrated circuit (ASIC) to implement the acquisition unit 211, first prediction unit 212, second prediction unit 213, output unit 214 and/or division unit 215. The acquisition unit 111, first prediction unit 212, second prediction unit 213, output unit 214 and/or division unit 215 may be implemented on a single integrated circuit or individually on a plurality of integrated circuits. The functions of the acquisition unit 211, first prediction unit 212, second prediction unit 213, output unit 214 and/or division unit 215 or programs for causing a computer to fulfill the functions may be recorded on a non-transitory computer-readable recording medium.

The division unit 215 divides a single genome region into a plurality of genome regions in accordance with a correlation between single-nucleotide polymorphisms on the basis of single-nucleotide polymorphism data of one individual. The division unit 215 divides a single genome region into a plurality of genome regions by a method common to a plurality of populations.

The first prediction unit 212 applies single-nucleotide polymorphism data to each of the first trait prediction models to calculate a plurality of first trait values for each of a plurality of genome regions.

The second prediction unit 213 calculates a second trait value for one individual on the basis of the first trait values calculated for each of the genome regions and a plurality of weighted average parameters which are associated with a population to which the individual belongs and which correspond to their respective first trait prediction models.

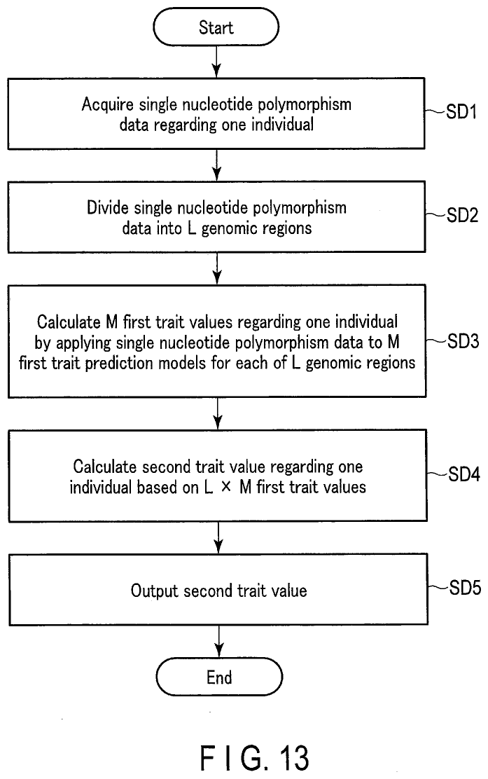

Next is a description of an example of a process of the trait prediction apparatus 2 according to the second embodiment. FIG. 13 is a flowchart of the example of a process of the trait prediction apparatus 2 according to the second embodiment. As shown in FIG. 13, first, the acquisition unit 211 acquires single-nucleotide polymorphism data over the genome regions for one individual whose trait is to be predicted (step SD1).

After step SD1, the division unit 215 divides the single-nucleotide polymorphism data acquired in step SD1 into L single-nucleotide polymorphism data corresponding to L genome regions, respectively (step SD2). In step SD2, the division unit 215 divides a genome region of the single-nucleotide polymorphism data acquired in step SD1, based on, for example, a division point on the top side and a division point on the bottom side of each of the genome regions defined by the division unit 116 of the trait prediction model generation apparatus 1. Thus, the single-nucleotide polymorphism data acquired in step SD1 is divided into L single-nucleotide polymorphism data corresponding to L genome regions, respectively. Note that the division unit 215 may divide a genome region by a method similar to that of the division unit 116 of the trait prediction model generation apparatus 1.

After step SD2, the first prediction unit 212 applies the single-nucleotide polymorphism to M first trait prediction models for each of the L genome regions to calculate M first trait values for the one individual whose trait is to be predicted (step SD3). After step SD3, the second prediction unit 213 calculates a second trait value for the one individual whose trait is to be predicted, based on the L×M first trait values calculated in step SD3 (step SD4). After step SD4, the output unit 214 outputs the second trait value calculated in step SD4 (step SD5). In step SD5, the output unit 214 may display the second trait value, for example, on the display device 25, record it in the storage device 22, or transmit it to another computer via the communication device 24.

When step SB4 is executed, the operation of the trait prediction apparatus 2 is terminated.

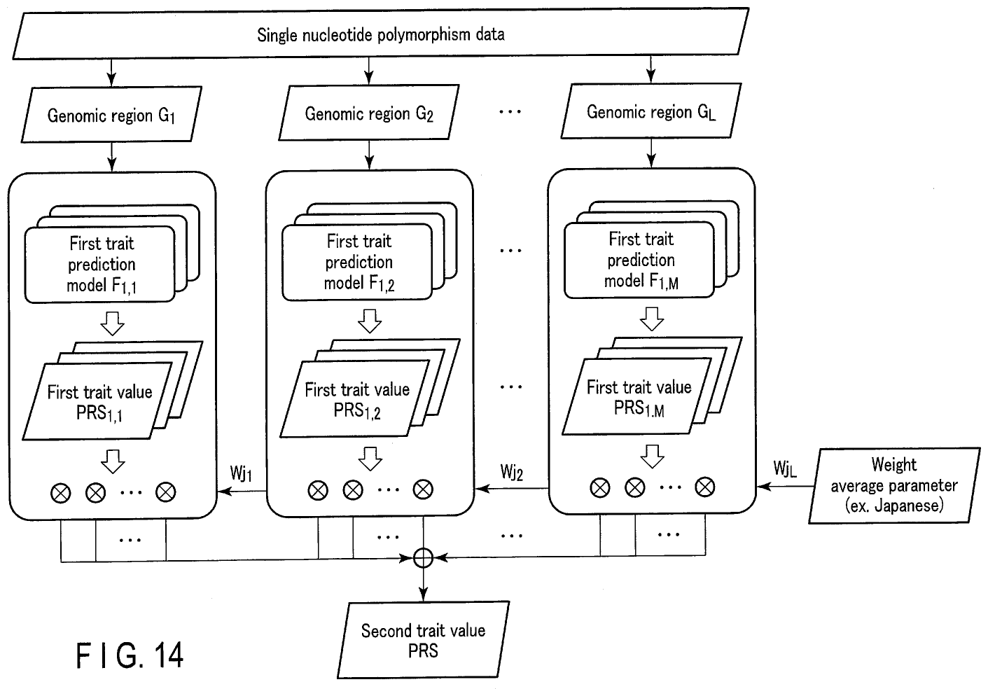

FIG. 14 is a schematic diagram of the example of a process of the trait prediction apparatus shown in FIG. 13. Assume that an individual whose trait is to be predicted is, for example, a Japanese. In this case, the first prediction unit 212 reads a second trait prediction model for the Japanese from the storage device 22. Specifically, the first prediction unit 212 selects and reads a second trait prediction model associated with an identifier corresponding to the Japanese from a plurality of second trait prediction models stored in the storage device 22. As the second trait prediction model corresponding to the Japanese, L×M first trait prediction models F_{m, l} and L×M weighted average parameters w_{m, l} are read out.

Then, the division unit 215 divides the single-nucleotide polymorphism data acquired in step SD1 into L single-nucleotide polymorphism data corresponding to L genome regions G₁. The first prediction unit 212 applies single-nucleotide polymorphism data of each of the genome regions G₁ to M first trait prediction models Fₘ to calculate M first trait values PRSₘ. Since the first trait values PRSₘ are calculated for all of the L genome regions G₁, L×M first trait values PRSₘ are calculated. Then, the second prediction unit 213 multiplies the L×M first trait values PRSₘ by the L×M weighted average parameters w_{m, l} in accordance with the following equation (11) to calculate L×M integrated values, and adds the L×M integrated values to calculate a second trait value PRS. It is thus possible to obtain a second trait value PRS with high accuracy for Japanese.

$$PRS = {\sum }_{m ∈ M} {\sum }_{l ∈ L} {w}_{{m}_{l}} {PRS}_{{m}_{l}} \qquad ( 11 )$$

As described above, according to the second embodiment, the second trait prediction model considering a difference in properties among the genome regions is used and thus a second trait value with higher prediction accuracy can be calculated.

Therefore, the foregoing embodiments improve the prediction accuracy of a trait of an individual.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A trait prediction model generation apparatus comprising a processing circuit configured to:
generate a plurality of first trait prediction models for each of a plurality of populations, based on summary statistics and inter-polymorphism correlated information; and
generate a second trait prediction model for a specific one of the populations based on regularized regression of the first trait prediction models of each of the populations using a plurality of data sets including single-nucleotide polymorphism data and a trait value.

2. The trait prediction model generation apparatus of claim 1, wherein the processing circuit generates the first trait prediction models using mutually different algorithms and a reference value for the summary statistics and/or the inter-polymorphism correlated information, based on the summary statistics and the inter-polymorphism correlated information.

3. The trait prediction model generation apparatus of claim 1, wherein:
the second trait prediction model is defined by a total sum of integrated values of an output value of each of the first trait prediction models and a weighted average parameter corresponding to the output value all over the populations and the first trait prediction models; and
the processing circuit determines, based on the plurality of data sets, a value of weight to minimize an objective function including a loss function between the output value and the trait value and a regularization term for the weighted average parameter.

4. The trait prediction model generation apparatus of claim 3, wherein the regularization term includes a sum of. an L1 regularization term and an L2 regularization term.

5. The trait prediction model generation apparatus of claim 1, wherein the processing circuit:
generates the first trait prediction models of each of the populations for each of a plurality of genome regions into which a genome region is divided in accordance with a correlation between single-nucleotide polymorphisms
generates the second trait prediction model based on the first trait prediction models generated for each of the genome regions of each of the populations.

6. The trait prediction model generation apparatus of claim 5, wherein the processing circuit divides a single genome region into a plurality of genome regions in accordance with a correlation between the single-nucleotide polymorphisms based on single-nucleotide polymorphism data for the populations.

7. The trait prediction model generation apparatus of claim 1, wherein the processing circuit acquires GWAS statistics as the summary statistics.

8. The trait prediction model generation apparatus of claim 1, wherein the processing circuit acquires a linkage disequilibrium coefficient as the inter-polymorphism correlated information.

9. A trait prediction apparatus comprises a processing circuit configured to:
acquire single-nucleotide polymorphism data on one individual;
apply the single-nucleotide polymorphism data to each of a plurality of trait prediction models to calculate a plurality of first trait values for the one individual;
calculate a second trait value for the individual based on the first trait values and a plurality of weighted average parameters which correspond to the trait prediction models, respectively and which are associated with a population to which the individual belongs; and
output the second trait value.

10. The trait prediction apparatus of claim 9, wherein:
the processing circuit divides the single-nucleotide polymorphism data into a plurality of pieces of division data corresponding to a plurality of genome regions, respectively, wherein,
the trait prediction models are provided for the genome regions, respectively;
the weighted average parameters are provided for the genome regions, respectively;
the processing circuit applies the division data corresponding to the genome regions to the trait prediction models to calculate the first trait values for each of the genome regions and output the second trait value based on the first trait values for the genome regions and the weighted average parameters.

11. A method for generating a trait prediction model, comprising:
generating a plurality of first trait prediction models for each of a plurality of populations, based on summary statistics and inter-polymorphism correlated information; and
generating a second trait prediction model for a specific one of the populations, based on regularized regression of the first trait prediction models of each of the populations using a plurality of data sets including single-nucleotide polymorphism data and a trait value.
